(19) United States
(12) Patent Application Publication
(10) Pub. No.: US 2020/0040326 A1
(43) Pub. Date: Feb. 6, 2020

(54) METHOD FOR PROVIDING A DNA-ENCODED LIBRARY, DNA-ENCODED LIBRARY AND METHOD OF DECODING A DNA-ENCODED LIBRARY

(71) Applicant: TU Dresden, Dresden (DE)

(72) Inventors: Yixin ZHANG, Dresden (DE); Francesco REDDAVIDE, Dresden (DE); Meiying CUI, Dresden (DE); Helena ANDRADE, Dresden (DE); Stephan HEIDEN, Dresden (DE)

(73) Assignee: TU Dresden, Dresden (DE)

(21) Appl. No.: 16/525,936

(22) Filed: Jul. 30, 2019

(30) Foreign Application Priority Data
Aug. 2, 2018 (EP) 18 186 948.8

(51) Int. Cl. C12N 15/10; C40B 40/08; C40B 50/10; C40B 70/00

(52) U.S. Cl. CPC C12N 15/1065; C40B 70/00; C40B 50/10; C40B 40/08

(57) ABSTRACT

Disclosed are a method for providing a DNA-encoding library, the DNA-encoding library and a method of decoding a DNA-encoded library. Many different DNA molecules are synthesized which differ from each other in DNA barcode sequences. Each DNA molecule is bonded to a specific substance forming different DNA-substance conjugates. The DNA-encoded library has the advantage that, for example after an enrichment experiment performed with the library, the library may be decoded in a faster and less expensive manner than known DNA-encoded libraries.

### CROSS-REFERENCE TO A RELATED APPLICATION

This patent application claims the benefit of European Patent Application No. 18 186 948.8, filed on Aug. 2, 2018, the disclosure of which is incorporated herein by reference in its entirety for all purposes.

### INCORPORATION BY REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,641 bytes ASCII (Text) file named “744446_ST25.txt”, created on Jul. 26, 2019.

A method for providing a DNA-encoding library, the DNA-encoding library and a method of decoding a DNA-encoded library are presented. Many different DNA molecules are synthesized which differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides. Each of the many different DNA molecules is bonded to at least a specific substance forming different DNA-substance conjugates, wherein the DNA-substance conjugates differ from each other by the specific substance and by their DNA molecules, wherein the first part and the third part encode information regarding the second part of the first coding region and wherein a certain first part and/or a certain third part uniquely codes for a certain group of DNA-substance conjugates which is smaller than the group of all DNA-substance conjugates in the DNA-encoded library. The DNA-encoded library has the advantage that, for example after an enrichment experiment performed with the library, the library may be decoded in a faster and less expensive manner than known DNA-encoded libraries.

In drug discovery which aims at identifying high affinity binders from a pool of molecules, it is known in the prior art to use a DNA-encoded library (“DEL”). Ideally, said DEL can mimic the function-information relationship of cells, such as T cells and B cells in adaptive immunity and peptide/protein-display technologies (e.g. phage display, ribosome display, yeast display). In T cells, B cells and/or phages, the functions (mediated e.g. by proteins expressed on cell surface) and associated information (coded e.g. by genetic information) are both confined in individual cells. The function-information relationship can be studied even if there is only a single copy of an individual cell presented in a given cell mixture.

A DEL is composed of a pool of different molecules, each being a conjugate between a small organic molecule and a specific DNA sequence (a so-called “DNA barcode”), thus realizing a direct physical connection between function (function of the small organic molecule by its chemical structure) and information (information about the type of small organic molecule coded by the DNA sequence). The DNA sequences are designed to identify the associated chemical structures using various technologies, e.g. Sanger sequencing, DNA array and/or high throughput sequencing.

Although PCR (polymerase chain reaction) is mainly used to amplify the selected compounds, PCR and real-time PCR (rtPCR) can also be used as a validation technique to check whether and at which abundance one particular DNA barcode is present, e.g. before and/or after the DEL has been subjected to a selection experiment. A selection experiment seeks to enrich certain conjugates between small organic molecules and DNA barcodes based on isolating said conjugates after they have bound to one or more desired target(s). Since the conjugates are enriched, a DEL selection experiment may be regarded as an experiment enriching certain DNA barcodes, namely those coding for small organic molecules having a high binding affinity to the target(s).

Similar like in the phage display technology, a usual DEL selection experiment provides tens to hundreds of DNA barcodes (DNA sequences) in one round of selection (one run). However, different from phage display technology, which regularly reveals organic molecules which are highly specific and potent binders (i.e. the k_{D} to the target lies in the pM to nM range), a DEL selection experiment frequently also reveals DNA barcodes coding for small organic molecules which are only moderate binders (e.g. the k_{D} to the target lies in the low to medium μM range).

In principle, Sanger sequencing provides a tool to decode DNA barcodes which have been found in a DEL selection experiment.

However, Sanger sequencing has the disadvantage that the throughput is low, i.e. the “reading” of the DNA barcode consumes a lot of time thus represents an uneconomical readout.

A further disadvantage of Sanger sequencing is its low sensitivity when analyzing complex mixtures of different DNA sequences. Assuming a DEL selection experiment using a DEL comprising 1 million different compounds, one compound is usually enriched 1000 times over the average and 100 sequences will be obtained from Sanger sequencing. In this case, there will be an approx. 90% chance that that one particular compound is not identified by the selection experiment, i.e. escapes identification, because its presence is not revealed by Sanger sequencing.

Moreover, even if a certain DNA barcode (e.g. coding for a certain small organic molecule) appears once in the enrichment process, Sanger sequence may identify said DNA barcode as coding for a small organic molecule which binds to the target. However, Sanger sequencing cannot reveal whether the identification of this specific small organic molecule has been a random event (i.e. an accidental hit) or is actually statistically significant (i.e. a true hit). In short, Sanger sequencing also suffers the disadvantage that false positives may not be distinguished from true positives without oversampling. While oversampling in the context of Sanger sequencing is apparently very important to obtain statistically meaningful results for hit identification in the decoding process (readout), it has become clear that Sanger sequencing is far from being efficient.

A DNA array provides an alternative solution to decode a DNA barcode sequence of binders identified in a DEL selection experiment. Since each DNA barcode sequence is associated with a certain physical location and evaluated according to its fluorescence intensity, the measurement avoids the requirement of oversampling using Sanger sequencing.

However, although fully complimentary sequences lead to highest signal intensity, strong background noise associated with mismatching DNA sequence interaction prevents the use of this method to decode a large library of DNA barcode sequences. For example, with a library of only few hundreds compounds each having a DNA barcode sequence, great effort needs to be made to distinguish a specific pair from mismatching and background noises. In short, the DNA array identification method also suffers the disadvantage that false positives may not be distinguished well from true positives. In other words, the systemic error of this identification method is high.

High throughput sequencing (“HTS”) has become the standard technology for decoding a DEL after a selection experiment. HTS applies a similar principle like Sanger sequencing and uses the count of a particular sequence as an indicator of its enrichment. Millions of sequence reads resulting from HTS make oversampling possible, even when a DEL of a relatively large size is used.

However, like the DNA array approach, HTS can only provide a semi-quantitative analysis of a selection experiment, because it was found that the counts of DNA barcode sequences and the measured affinity of its bound small organic molecule to the desired target(s) only show a poor correlation. The identified poor correlation has not yet been fully understood. Principally, it could be caused by a low synthetic quality of the DNA barcodes, while biases during the PCR and sequencing process may play a role. In summary, HTS is prone to reveal many false positive hits during the identification process, i.e. the systemic error of this identification method is high.

Moreover, as the size of a DEL has been increasing gradually in recent years, HTS will no longer fulfill the requirement of oversampling when a DEL has started to comprise billions of compounds.

Furthermore, although HTS has become cheaper in the last years, it is still very expensive for many academic researchers. The outsourced sequencing tasks normally take a few weeks while researchers have no control over the sequencing experiments.

PCR and rtPCR have been used in the prior art to overcome the problems of the Sanger sequencing, DNA array and HTS identification methods. The advantages of both PCR and rtPCR are that primer pairs can be designed for a certain code. In other words, different primers may be used which themselves can carry a “code” in the sense that some of the primers bind (at least partially) to certain codes and some other do not. Additionally, rtPCR has the advantage over PCR that it will reveal a difference between a positive control and a negative control (in real time) and thus allows a better discrimination between false and true positives.

However, although rtPCR provides a quantitative analysis of a DEL selection process, it can only be designed for a limited number of codes and compounds. Therefore rtPCR suffers the disadvantage that it cannot be used for decoding the results of de novo selection experiments.

Starting herefrom, it was the object of the present invention to provide a method for encoding and decoding DNA barcodes having been enriched in a selection experiment with a DNA encoded library, wherein the method shall overcome the deficiencies of the prior art identification methods. Specifically, the method should be a facile, cost-efficient, quantitative, highly sensitive (i.e. be capable for revealing also weak binders), highly specific (i.e. be capable to reveal more true positives than false positives) and suitable to decode de novo selection experiments.

The object is solved by the method for providing a DNA-encoded library described herein, the DNA-encoded library described herein, and the method of decoding said DNA-encoded library described herein, as well as the advantageous embodiments thereof.

According to the invention, a method for providing a DNA-encoded library (DEL) is provided, the method comprising
a) synthesizing many different DNA molecules which differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides; and
b) bonding each of the many different DNA molecules to at least a specific substance forming different DNA-substance conjugates, wherein the DNA-substance conjugates differ from each other by the specific substance and by their DNA molecules;

characterized in that the first part and the third part encode information regarding the second part of the first coding region, wherein a certain first part and/or a certain third part uniquely codes for a certain group of DNA-substance conjugates which is smaller than the group of all DNA-substance conjugates in the DNA-encoded library.

The advantage of the DNA-encoded library (“DEL”) provided by the inventive method is that both the first and third part of the DNA barcode sequence each encode for a certain subgroup of DNA-substance conjugates within the DEL. In qPCR, a primer binding to the first part of the DNA barcode sequence will give a strong signal (strong amplification) if the subgroup of DNA-substance conjugates for which the first part encodes (e.g. transcription factors) has been enriched in a previous selection experiment performed with the DEL. The same is true for the third part of the DNA barcode sequence, i.e. a primer binding to the third part of the DNA barcode sequence will give a strong signal (strong amplification) if the subgroup of DNA-substance conjugates for which the third part encodes (e.g. zinc finger proteins) has been enriched in a previous selection experiment performed with the DEL. If strong signal is obtained for both a primer binding to the first part and a primer binding to the third part after qPCR, the skilled person knows that DNA-substance conjugates belonging to both subgroups (e.g. zinc finger transcription factors) have been strongly enriched. The skilled person obtains this information only via qPCR with the inventive DEL and suitable primers, i.e. the skilled person does not have to perform a DNA sequencing. This allows a much faster and less expensive decoding of a DNA-encoded library after a selection experiment performed with said library.

The DNA-encoded library can be used to construct many two-dimensional matrices in which different first primers which bind to different first parts of the barcode form the rows of the matrix, different second primers which bind to different second parts of the barcode are the columns of the matrix and the signal intensity after qPCR with each primer pair is given in each field of the matrix (crossing point between rows and columns). The signal intensity obtained for each primer pairing allows a deconvolution of the mixture of DNA barcodes, i.e. of the DEL after the selection experiment. The possibility to deconvolute the mixture of DNA barcodes strongly improves the specificity of the identification method, i.e. its capability of distinguishing true positive hits from false positive hits and allows a quick determination of “hits” even without performing DNA sequencing.

Since performing qPCR without DNA sequencing is not expensive, it is estimated that a full decoding experiment will cost only approx. 50 €. Thus, the DEL produced with the inventive method allows a very cost-efficient “hit” detection after an enrichment experiment with said DEL and needs very little investment in instrumentation. Additionally, the DEL allows to obtain a more quantitative information on the abundance of a certain DNA barcodes after a selection experiment as compared with previously known DELs.

The inventive method can be characterized in that
i) the first coding region DNA sequence comprises at least a fourth part, wherein the second part is located between the fourth and third part and wherein both the combination of the first part and the fourth part and the combination of the first part and the third part of the first coding region encode information about the second part of the first coding region; and
ii) each barcode sequence comprises at least a second coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part of the second coding region encode information about the second part of the second coding region;

wherein a certain combination of a first part and fourth part in a certain coding region uniquely codes for a certain group of DNA-substance conjugates which is smaller than the group of all DNA-substance conjugates which is encoded by the first part alone.

In this embodiment of the invention, the DNA-encoded library can be used to construct more two-dimensional matrices because an additional primer can be used which anneals to the fourth part of the DNA barcode and because a further coding region with different four parts is present. Only with one single run of qPCR, very detailed information is obtained about the specific groups of DNA-substance conjugates that have been enriched in the selection experiment with the DEL.

Furthermore, the inventive method can be characterized in that
i) each barcode sequence comprises at least a second coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part of the second coding region encode information about the second part of the second coding region; and
ii) each barcode sequence comprises at least a third coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and third part and the of the third coding region encode information about the second part of the third coding region;

wherein a certain combination of a first part and fourth part in a certain coding region uniquely codes for a certain group of DNA-substance conjugates which is smaller than the group of DNA-substance conjugates which is encoded by the first part.

In view of the further coding region and the separation of at least one coding region into five parts, more different primers can be used in one single qPCR and within one single run of qPCR, very detailed information can obtained which specific groups of DNA-substance conjugates have been enriched in the selection experiment with the DEL.

In a preferred embodiment of the invention, at least one coding region DNA sequence, optionally all coding region DNA sequences, comprise at least a first part, a second part, a third part, a fourth part and a fifth part, wherein the second part is located between the fourth and fifth part and the second part differs between all the DNA molecules by at least two nucleotides, wherein the combination of the first part and the fourth part and the combination of the fifth part and the third part of the coding region encode information about the second part of the coding region, preferably of all coding regions, wherein a certain combination of a first part and fourth part uniquely codes for a certain group of DNA-substance conjugates which is smaller than the group of DNA-substance conjugates which is encoded by the first part alone, and wherein a certain combination of a fifth part and third part uniquely codes for a certain group of DNA-substance conjugates which is smaller than the group of DNA-substance conjugates which is encoded by the third part alone.

Since in this embodiment, at least one coding region has not three or four, but actually five parts, a total of four primers can be used in each qPCR for amplifying the at least one coding region. In short, one primer annealing to the first part, one primer annealing to the third part, one primer annealing to the fourth part and one primer annealing to the fifth part can be used. This gives a total amount of 6 two-dimensional matrices. Thus, in one single qPCR, more detailed information is obtained which specific groups of DNA-substance conjugates have been enriched in the selection experiment with the DEL.

Furthermore, according to the invention, a DNA-encoded library is provided. The DNA-encoded library comprises many different DNA-substance conjugates, wherein the DNA-substance conjugates differ from each other by their substance and by their DNA molecules, wherein the DNA molecules of the DNA-substance conjugates differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides, characterized in that the first part and the third part encode information regarding the second part of the first coding region, wherein a certain first part and/or a certain third part uniquely codes for a certain group of DNA-substance conjugates which is smaller than the group of all DNA-substance conjugates in the DNA-encoded library.

The inventive DNA-encoded library can be characterized in that
i) the first coding region DNA sequence comprises at least a fourth part, wherein the second part is located between the fourth and third part and wherein both the combination of the first part and the fourth part and the combination of the first part and the third part of the first coding region encode information about the second part of the first coding region; and
ii) each barcode sequence comprises at least a second coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part of the second coding region encode information about the second part of the second coding region;

wherein a certain combination of a first part and fourth part in a certain coding region uniquely codes for a certain group of DNA-substance conjugates which is smaller than the group of all DNA-substance conjugates which is encoded by the first part alone.

Furthermore, the inventive DNA-encoded library can be characterized in that ach barcode sequence comprises at least a third coding region DNA sequence, which is on the same DNA strand as the second coding region, comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part and the of the third coding region encode information about the second part of the third coding region, wherein a certain combination of a first part and fourth part in the second coding region and in the third coding region uniquely codes for a certain group of DNA-substance conjugates which is smaller than the group of DNA-substance conjugates which is encoded by the first part alone.

In a preferred embodiment of the invention, the DNA-encoded library is characterized in that at least one coding region DNA sequence, optionally all coding region DNA sequences, comprise at least a first part, a second part, a third part, a fourth part and a fifth part, wherein the second part is located between the fourth and fifth part and the second part differs between all the DNA molecules by at least two nucleotides, wherein the combination of the first part and the fourth part and the combination of the fifth part and the third part of the coding region encode information about the second part of the coding region, preferably of all coding regions, and wherein a certain combination of a first part and fourth part uniquely codes for a certain group of DNA-substance conjugates which is smaller than the group of DNA-substance conjugates which is encoded by the first part alone and wherein a certain combination of a fifth part and third part uniquely codes for a certain group of DNA-substance conjugates which is smaller than the group of DNA-substance conjugates which is encoded by the third part alone.

In a further preferred embodiment, the DNA-encoded library is producible or produced by the inventive method for providing a DNA-encoded library,

Moreover, according to the invention, a method of decoding the inventive DNA-encoded library is provided. The method comprises
a) performing a qPCR with the DNA-encoded library according to one of claims 5 to 8 as template, wherein the following primers are used:
   a primer A and a primer B for amplifying the first coding region of every DNA-substance conjugate; and
   many different primers A-xN which anneal to the different first parts of the first coding region and many different primers B-yN which anneal to the different third parts of the first coding region, wherein primer A-xN has an identical length like the coding region primer A by shortening x nucleotides at its 5′-end, primer B-yN has an identical length like the coding region primer B by shortening y nucleotides at its 5′-end, N represents a A, T, G or C and x and y represent the total number of any one of A, T, G or C at the 3′-end of the primers, wherein x is an integer from 2 to 6, preferably 4;
b) calculating a mathematical product of the signal value of each primer A-xN and each primer B-xN by following equation:

Value (A-xN)ᵢ=signal value [(A-xN)_{i+B}]·signal value [(A-xN)ᵢ+(B-xN)ᵢ]; and

Value (B-yN)ᵢ=signal value [(B-yN)_{i+A}]·signal value [(B-yN)ᵢ+(A-xn)ᵢ],
wherein i is an integer and defines a specific primer, and the “+”-sign indicates a combination of two primers; wherein signal value is the percentage of abundance related to the whole set of qPCR quantification using different primers annealed to the same region; and
c) comparing the obtained mathematical products for each of the primers (A-xN)ᵢ and (B-yN)ᵢ, wherein those primers with high values code for DNA-substance conjugates which are present at a high concentration in the DNA-encoded library.

The method of decoding the inventive DNA-encoded library can be characterized in that the method comprises
i) calculating a mathematical product of the value obtained for each primer A-xN and each primer B-yN by following equation

Value(A−B)ᵢ=Value(A-xN)ᵢ·Value(B-yN)ᵢ;
II) comparing the obtained mathematical products for each of the combination of primers (A-xN)ᵢ and (B-yN)ᵢ, wherein those primer combinations with high values code for DNA-substance conjugates which are present at a high concentration in the DNA-encoded library.

Furthermore, the method of decoding the inventive DNA-encoded library can be characterized in that the qPCR is performed with the inventive DNA-encoded library according and the method comprises
I) performing a qPCR with the following primers:
   a first coding region primer A and a first coding region primer primer B for amplifying the first coding region of every DNA-substance conjugate; and
   many different primers A-xN which anneal to the different first parts, or first and fourth parts of the first coding region and many different primers B-yN which anneal to the different third parts of the first coding region, wherein A-xN has an identical length like the coding region primer A by shortening x nucleotides at its 5′-end, B-yN has an identical length like the coding region primer B by shortening y nucleotides at its 5′-end, N represents a A, T, G or C and x and y represent the total number of any one of A, T, G or C at the 3′-end of the primers, wherein x is an integer from 6 to 10, preferably 8, and y is an integer from 2 to 6, preferably 4; and
   a second coding region primer C and a second coding region primer D for amplifying the second coding region of every DNA-substance conjugate; and
   many different primers D-yN which anneal to the different first parts, or first and fourth parts of the second coding region and many different primers C-xN which anneal to the different third parts of the second coding region, wherein primer C-xN has an identical length like the coding region primer C by shortening x nucleotides at its 5′-end, primer D-yN has an identical length like the coding region primer D by shortening y nucleotides at its 5′-end, N represents a A, T, G or C and x and y represent the total number of any one of A, T, G or C at the 3′-end of the primers, wherein x is an integer from 6 to 10, preferably 8, and y is an integer from 2 to 6, preferably 4;
II) calculating a mathematical product of the signal value of each primer A-xN, each primer B-yN, each primer C-xN and each primer D-yN by following equation:

Value (A-xN)ᵢ=signal value [(A-xN)ᵢ+B]·signal value [(A-xN)ᵢ+(B-xN)ᵢ];

Value (B-yN)ᵢ=signal value [(B-yN)ᵢ+A]·signal value [(B-yN)ᵢ+(A-xN)ᵢ],

Value (C-xN)ᵢ=signal value [(C-xN)ᵢ+D]·signal value [(C-xN)ᵢ+(D-xn)ᵢ],

Value (D-yN)ᵢ=signal value [(D-yN)ᵢ+C]·signal value [(D-yN)ᵢ+(C-xn)ᵢ],
wherein i is an integer and defines a specific primer, and the “+”-sign indicates a combination of two primers; wherein signal value is the percentage of abundance related to the whole set of qPCR quantification using different primers annealed to the same region; and
III) comparing the obtained mathematical products for each of the primers (A-xN)ᵢ, (By-N)ᵢ, (C-xN)ᵢ and (D-yN)ᵢ, wherein those primers with high values code for DNA-substance conjugates which are present at a high concentration in the DNA-encoded library.

In a preferred embodiment of the invention, the method of decoding the inventive DNA-encoded library comprises
I) calculating a mathematical product of the value obtained for each primer A-xN and each primer B-yN, for each primer A-xN and each primer D-xN and for each primer C-yN and D-xN by following equation

Value (A−B)ᵢ=Value (A-xN)ᵢ·Value (B-yN)ᵢ;

Value (A−D)ᵢ=Value (A-xN)ᵢ·Value (D-yN)ᵢ;

Value (C−D)ᵢ=Value (C-xN)ᵢ·Value (D-yN)ᵢ;
II) calculating the mathematical product of the Value (A−B)ᵢ, (A−D)ᵢ and (C−D)ᵢ for each primer i by the following equation

Valueⁱ=value(A−B)ᵢ·value(A−D)ᵢ·value(C−D)ᵢ
III) comparing the obtained mathematical products Valueⁱ, wherein those primer combinations i with high values code for DNA-substance conjugates which are present at a high concentration in the DNA-encoded library.

In a further preferred embodiment, the method of decoding the inventive DNA-encoded library is characterized in that the qPCR is performed with the inventive DNA-encoded library as template and the method comprises
I) performing a qPCR with the following primers:
   a first coding region primer A and a first coding region primer B for amplifying the first coding region of every DNA-substance conjugate; and
   many different primers A-xN which anneal to the different first parts, or first and fourth parts of the first coding region, and many different primers B-yN which anneal to the different third parts of the first coding region, wherein A-xN has an identical length like the coding region primer A by shortening x nucleotides at its 5′-end, B has an identical length like the coding region primer B-yN by shortening y nucleotides at its 5′-end, N represents a A, T, G or C and x and y represent the total number of any one of A, T, G or C at the 3′-end of the primers, wherein x is an integer from 6 to 10, preferably 8, and y is an integer from 2 to 6, preferably 4; and
   a second coding region primer C and a second coding region primer D for amplifying the second coding region of every DNA-substance conjugate; and
   many different primers D-yN which anneal to the different first parts, or first and fourth parts of the second coding region and many different primers C-xN which anneal to the different third parts of the second coding region, wherein primer C-xN has an identical length like the coding region primer C by shortening x nucleotides at its 5′-end, primer D-yN has an identical length like the coding region primer D by shortening y nucleotides at its 5′-end, N represents a A, T, G or C and x and y represent the total number of any one of A, T, G or C at the 3′-end of the primers, wherein x is an integer from 6 to 10, preferably 8, and y is an integer from 2 to 6, preferably 4;
   a third coding region primer E and a third coding region primer F for amplifying the third coding region of every DNA-substance conjugate; and many different primers E-xN which anneal to the different first parts of the third coding region and many different primers F-yN which anneal to the different third parts of the third coding region, wherein primer E-xN has an identical length like the coding region primer E by shortening x nucleotides at its 5′-end, primer F-yN has an identical length like the coding region primer F by shortening y nucleotides at its 5′-end, N represents a A, T, G or C and x and y represent the total number of any one of A, T, G or C at the 3′-end of the primers, wherein x is an integer from 6 to 10, preferably 8, and y is an integer from 2 to 6, preferably 4;
II) calculating a mathematical product of the signal value of each primer A-xN, each primer B-yN, each primer C-xN, each primer D-yN, each primer E-xN and each primer F-yN by following equation:

Value (A-xN)ᵢ=signal value [(A-xN)ᵢ+B]·signal value [(A-xN)ᵢ+(B-xN)ᵢ];

Value (B-yN)ᵢ=signal value [(B-yN)ᵢ+A]·signal value [(B-yN)ᵢ+(A-xN)ᵢ],

Value (C-xN)ᵢ=signal value [(C-xN)ᵢ+D]·signal value [(C-xN)ᵢ+(D-xN)ᵢ],

Value (D-yN)ᵢ=signal value [(D-yN)ᵢ+C]·signal value [(D-yN)ᵢ+(C-xN)ᵢ],

Value (E-xN)ᵢ=signal value [(E-xN)ᵢ+F]·signal value [(E-xN)ᵢ+(F-xn)ᵢ],

Value (F-yN)ᵢ=signal value [(F-yN)ᵢ+E]·signal value [(F-yN)ᵢ+(E-xN)ᵢ],
wherein i is an integer and defines a specific primer, and the “+”-sign indicates a combination of two primers; wherein signal value is the percentage of abundance related to the whole set of qPCR quantification using different primers annealed to the same region; and
III) comparing the obtained mathematical products for each of the primers (A-xN)ᵢ, (B-yN)ᵢ, (C-xN)ᵢ, (D-yN)ᵢ, (E-xN)ᵢ and (N-yN)ᵢ, wherein those primers with high values code for DNA-substance conjugates which are present at a high concentration in the DNA-encoded library.

The method of decoding the inventive DNA-encoded library may comprise
I) calculating a mathematical product of the value obtained for each primer A-xN and each primer B-yN, for each primer A-xN and each primer D-xN, for each primer C-yN and D-xN, for each primer A-xN and N-yN, for each primer M-xN and D-yN and for each primer M-xN and N-yN by following equation

Value (A−B)ᵢ=Value (A-xN)ᵢ Value (B-yN)ᵢ;

Value (A−D)ᵢ=Value (A-xN)ᵢ Value (D-yN)ᵢ;

Value (C−D)ᵢ=Value (C-xN)ᵢ Value (D-yN)ᵢ;

Value (A−F)ᵢ=Value (A-xN)ᵢ Value (F-yN)ᵢ;

Value (E−D)ᵢ=Value (E-xN)ᵢ Value (D-yN)ᵢ;

Value (E−F)ᵢ=Value (E-xN)ᵢ Value (F-yN)ᵢ;
II) calculating the mathematical product of the values (A−B)ᵢ, (A−D)ᵢ, (C−D)ᵢ, (A-F)ᵢ, (E-D)ᵢ and (E-F)ᵢ for each primer combinations i by the following equation

Valueⁱ=value (A−B)ᵢ·value (A−D)ᵢ·value (C−D)ᵢ·value (A−F)ᵢ·value (E−D)ᵢ·value (E−F)ᵢ;
III) comparing the obtained mathematical products Valueⁱ, wherein those primer combinations i with high values code for DNA-substance conjugates which are present at a high concentration in the DNA-encoded library.

In a preferred embodiment, the method is characterized in that it comprises the calculation of a Value^{i′} by the following calculation:

Value^{i′}=log₁₀[value (A−B)ᵢ·value (A−D)ᵢ·value (C−D)ᵢ·value (A−F)ᵢ·value (E−D)ᵢ·value (E−F)ᵢ].

With reference to the following Figures and Examples, the subject according to the invention is intended to be explained in more detail without wishing to restrict said subject to the special embodiments shown here.

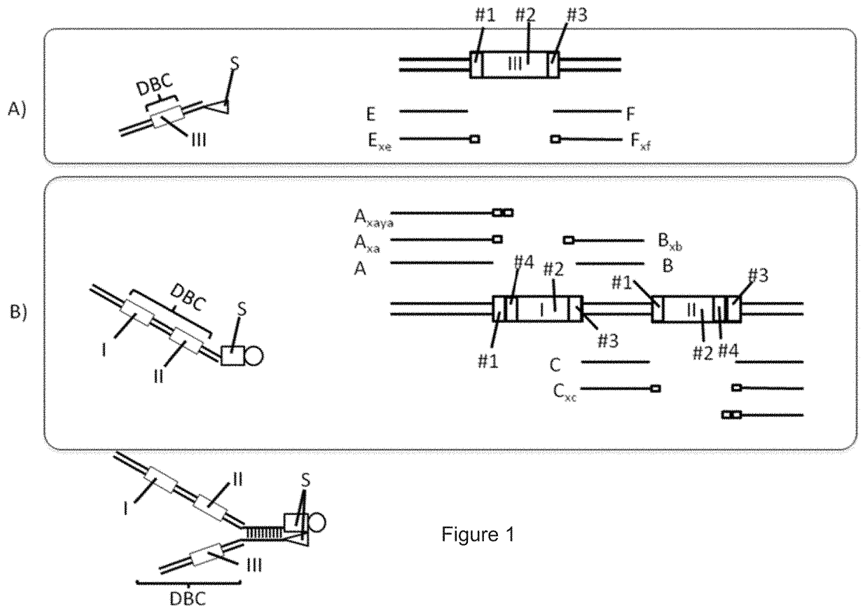

FIG. 1A shows how the coding algorithm works for generating a qPCR-matrix for DNA codes 1 having one single coding region 2 (coding region III). E and F are primary primers and Eₓₑ and F_{xf} are secondary primers. Primary primer E binds upstream (i.e. towards the 5′-end) of the first region #1 and primary primer F binds upstream (i.e. towards the 5′-end) of the third region #3. A qPCR with only the two primary primers E, F amplifies the DNA barcodes of all DNA-substance conjugates of the DNA-encoded library having coding region III. A qPCR with at least one primary primer E, F and at least one secondary primer Eₓₑ, F_{xf} is called a “primary qPCR”. FIG. 1A illustrates a qPCR template containing one single coding region III having the three code parts (sub-codes) #1, #2, #3. The sequence of the second part #2 of the coding region III is a unique sub-code. Each combination of the first part #1 and the third part #3 can also represent a unique code. Therefore, a sequence of the second part #2 is corresponding to a combination of the first part #1 and the third part #3. For each code part (sub-code) #1, #2, #3, there is a minimal difference number n between any pair of sequences (e.g. between two different xe sequences), while n should be ≥2. This means that the code parts #1, #2, #3 differs from each other by at least two nucleotides.

FIG. 1B shows how the coding algorithm works for generating a qPCR-matrix for DNA codes having two coding regions, namely coding region I and coding region II. A, B, C and D are primary primers, Aₓₐ, B_{xb}, C_{xc} and D_{xd} are secondary primers and A_{xaya} and D_{xdyd} are tertiary primers. A qPCR comprising the use of at least two tertiary primers is called a “tertiary PCR”.

FIG. 1B illustrates a qPCR template containing two different coding regions I, II, wherein the first coding region I has four code parts (sub-codes) #1, #2, #3, #4 and the second coding region II also has four code parts (sub-codes) #1, #2, #3, #4. The sequence of the second parts #2 of each coding region I, II represents a unique sub-code. Each combination of the first part #1 and the third part #3 of each coding region I, II can also represent a unique sub-code of each coding region I, II. In this case, a sequence of the second part #2 of each coding region I, II is corresponding to a combination of the sequence of the first part #1 and the sequence of the third part #3 of each coding region I, II. Each combination of the first part #1 the fourth part #4 can also represent one unique building block. In this case, a sequence of the second part #2 of each coding region I, II is also corresponding to a combination of the sequence of the first part #1 and the sequence of the fourth part #4 of each coding region I, II. For each code part (sub-code) #1, #2, #3, #4, there is a minimal difference number n between any pair of sequences (e.g. between two different #2 sequences), while n should be ≥2. This means that each code part #1, #2, #3, #4 differs from another code part #1, #2, #3, #4 by at least two nucleotides.

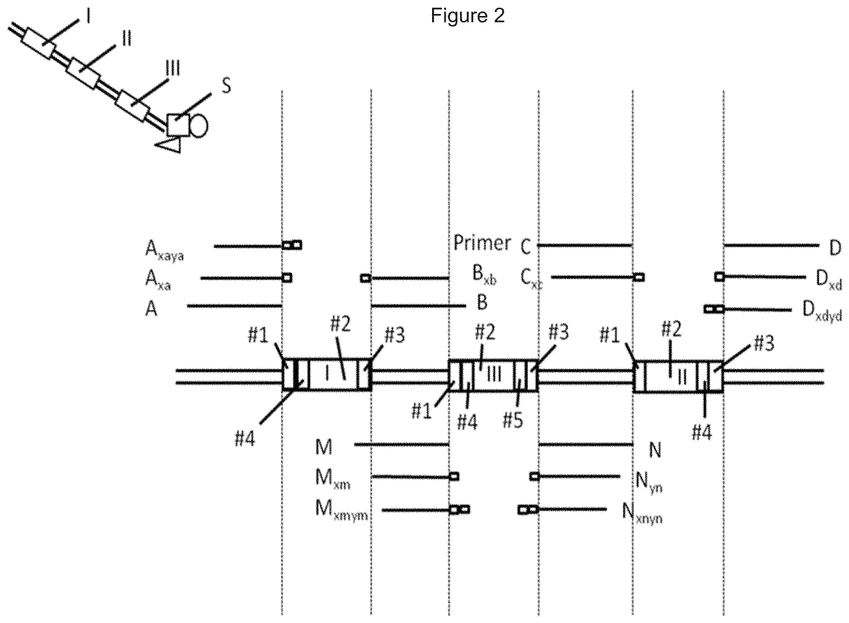

FIG. 2 shows how the coding algorithm works for generating a qPCR-matrix for DNA codes having three coding regions I, II, III. A, B, C and D are each a primary primer. Aₓₐ, B_{xb}, C_{xc}, D_{xd}, Mₓₘ and Nₓₙ are each a secondary primer. A_{xaya}, D_{xdyd}, M_{xmym} and N_{xnyn} are each a tertiary primer. A qPCR using at least two tertiary primers is called a “tertiary PCR”. FIG. 2 illustrates a qPCR template containing three different coding regions I, II, III, wherein the first coding region I has four code parts (sub-codes) #1, #2, #3, #4, the second coding region II also has four code parts (sub-codes) #1, #2, #3, #4 and the third coding region III has five code parts (sub-codes) #1, #2, #3, #4, #5. The sequence of each second code part #2 of each coding region I, II, III is a unique sub-code. Each combination of code parts #1 and code part #3, code part #1 with code part #4 and code part #1 with code part #5 can also represent a unique sub-code. For example, a sequence of code part #2 is corresponding to a combination of code part #1 and code part #3. For each code part (sub-code), there is a minimal difference number n between any pair of sequences (e.g. between two different ab sequences), while n should be ≥2. This means that each code part #1, #2, #3, #4, #5 differs from another code part #1, #2, #3, #4, #5 by at least two nucleotides.

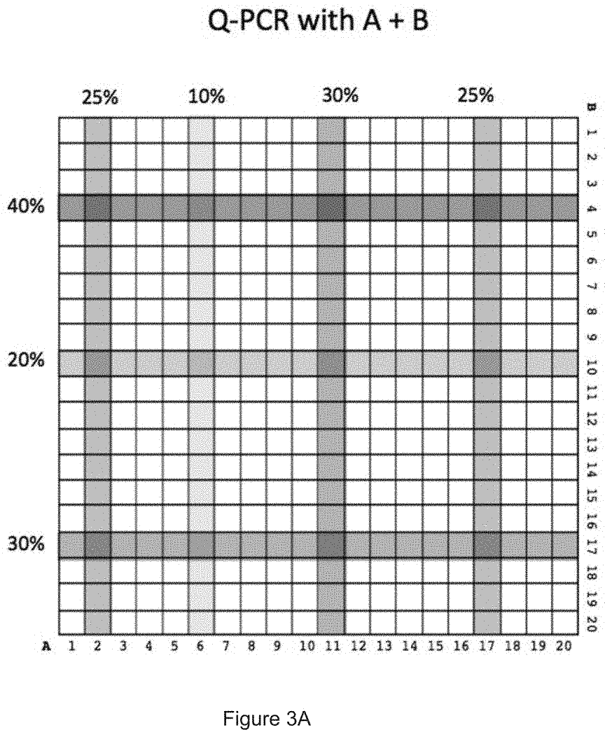

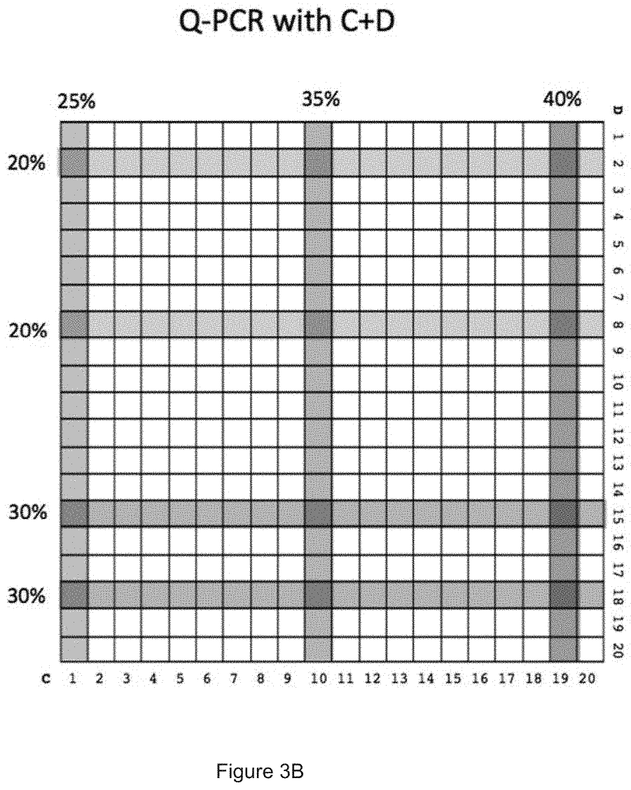

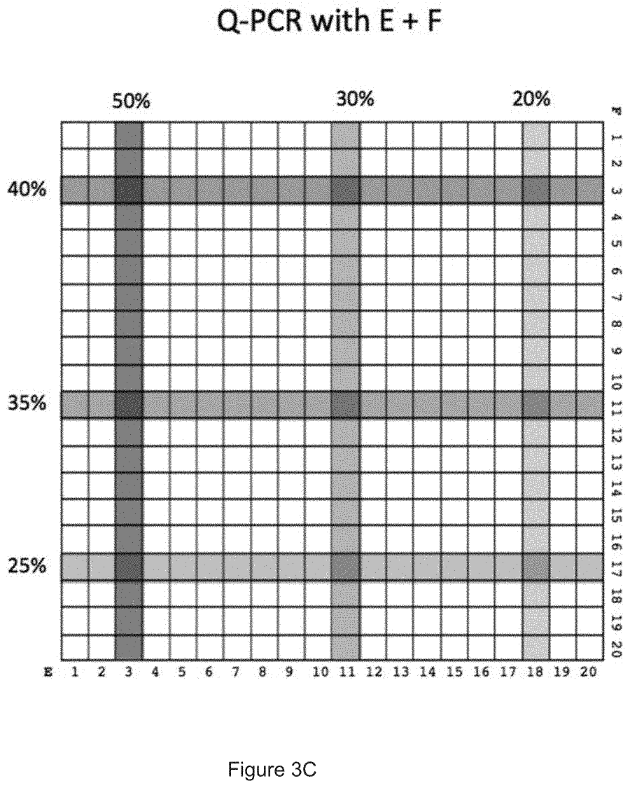

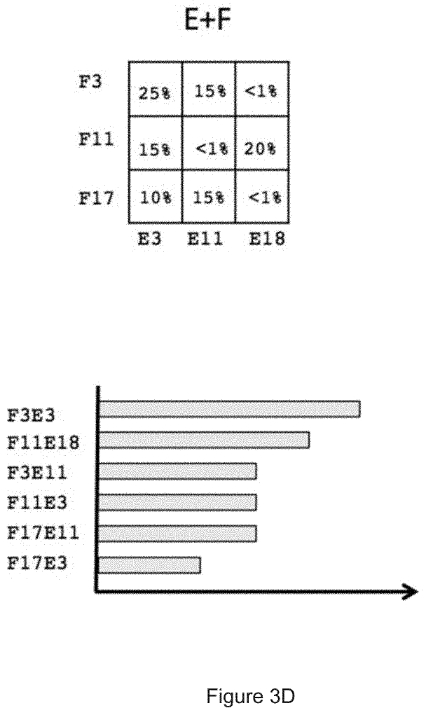

FIGS. 3A, 3B and 3C show three different qPCR matrices which were obtained after a qPCR using a DNA barcode having three coding regions I, II, III as template and 20 different primary primers A and 20 different primary primers B for binding to coding region I (see matrix “QPCR with A+B” in FIG. 3A, columns=different primers A, lines=different primers B), 20 different primary primers C and 20 different primary primers D for coding region II (see matrix “Q-PCR with C+D” in FIG. 3B, columns=different primers C, lines=different primers D) and 20 different primary primers E and 20 different primary primers F for coding region III (see matrix “Q-PCR with E+F” in FIG. 3C, columns=different primers E, lines=different primers F). An exemplary result of the matrix is illustrated in the table “E+F” in FIG. 3D which the primer pairs with the strongest amplification signal are listed together with their obtained (normalized) amplification signal. It can be derived from said table that the strong amplification signals have been obtained with the primer pairs E3 and F3 (25%), E18 and F11 (20%), E3 and F11 (15%), E11 and F3 (15%) and E11 and F17 (15%) and a medium amplification signal has been obtained with the primer pair E3 and F17 (10%). Below the table “E+F” in FIG. 3D, the obtained result is also shown in a column diagram. It can be derived from the obtained result that DNA-substance conjugates to which e.g. the primer pair E3 and F3 binds had a high concentration in the DNA-encoded library (after the enrichment experiment) and DNA-substance conjugates to which e.g. the primer pair E3 and F17 binds had a lower concentration in the DNA-encoded library (after the enrichment experiment). It may also be concluded that DNA-substance conjugates to which primer pairs with no signal (e.g. E1 and F1) bind were not present in the DNA-encoded library (after the enrichment experiment). Since the substance connected to each specific DNA coding region is known, this approach allows a fast and sensitive identification of substances being present at a high concentration after a (DEL) selection experiment.

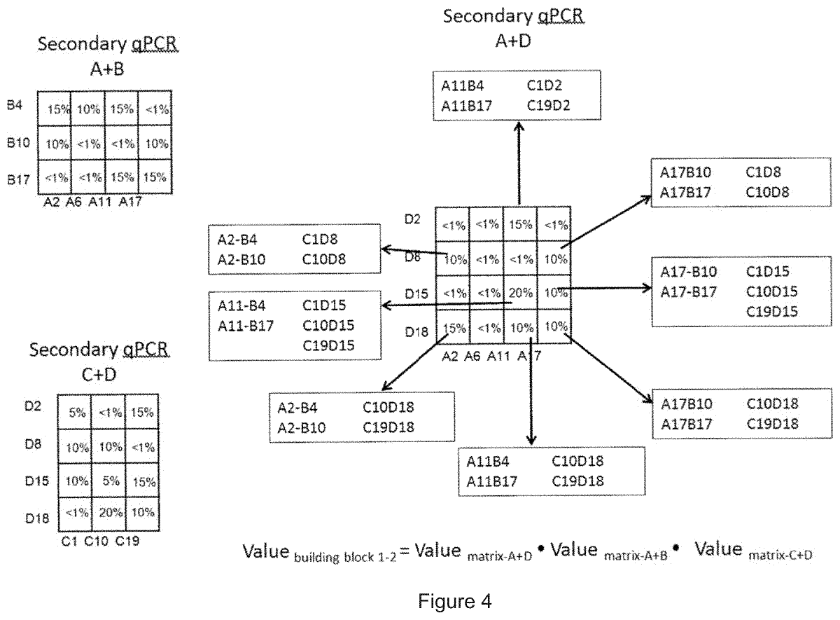

FIG. 4 shows the results of the qPCR matrices “Q-PCR with A+B” (amplification of coding region I) and “Q-PCR with C+D” (amplification of coding region II) from FIG. 3 and also the results of a secondary PCR with primer pairs A and D (coding region between I and II; see FIG. 2). qPCR with the primer pair A+D gave strong amplification signals for the specific primer pairs A11 and D15 (20%), A11 and D2 (15%), A2 and D18 (15%) and medium amplification signals for the primer pairs A2 and D8 (10%), A11 and D18 (10%), A17 and D18 (10%), A17 and D15 (10%) and A17 and D8 (10%). Each of said identified A primers binds to a specific coding region I and each of said identified D primers binds to a specific coding region II. This means that the primers A and D which gave strong signals code for coding regions I and II which must have been enriched in the DNA-encoded library after the enrichment experiment. It can also be concluded that the two coding regions I and II must be located on one single DNA strand because otherwise, no amplification signal would have been obtained. In order to combine the result obtained with the primer pair A and D with the result of the other primer pairs A and B and C and D, the mathematical product of the value obtained for each specific primer pair is calculated by the equation Value_{coding region I-II}=Value_{matrix-A+D}·Value_{matrix-A+B}·Value_{matrix-C+D}. Specific primers A, B, C and D which resulted in a high amplification signal consequently have a high Value_{coding region I-II}. Thus, the obtained Value_{coding region I-II} allows the identification of primers which must have bound to abundant DNA-barcodes and thus allows the identification of substances (bound to the DNA-barcode) which were abundantly present after the (DEL) selection experiment.

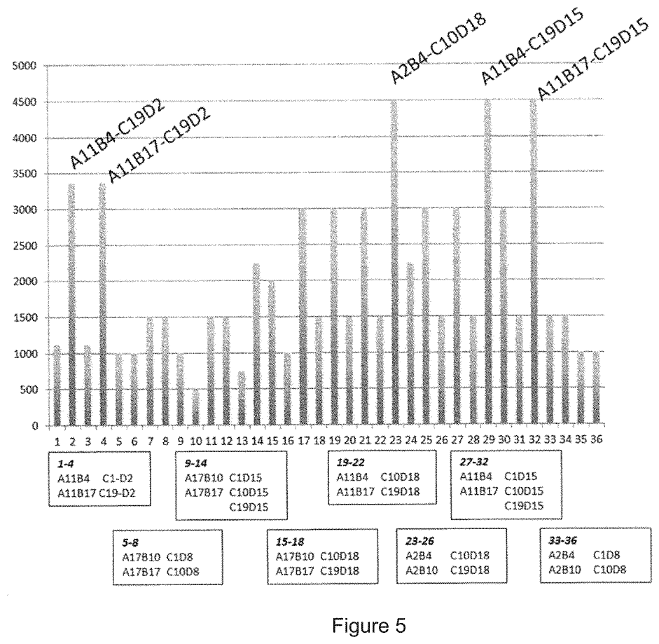

FIG. 5 shows a plot of 36 different combinations of coding regions I (A+B) and II (C+D) which gave the highest mathematical product according to equation Value_{coding region I-II}=Value_{matrix-A+D}·Value_{matrix-A+B}·Value_{matrix-C+D} (see absolute value in arbitrary unit on y-axis). In said plot, it can be visually identified that the combinations of coding region I and II with the numbers 2, 4, 23, 29 and 32 on the x-axis achieved the highest score. These numbers refer to the following five different combinations of coding regions I and II: A11B4-C19D2 (no. 2), A11B17-C 19D2 (no. 4), A2B4-C10-D18 (no. 23), A11B4-C19D15 (no. 29) and A11B17-C19D15 (no. 32). Naturally, it is known for which substances (or plurality of substances) these five different combinations encode. Thus, it is possible to identify five different (groups of) substances which have been strongly enriched in a (DEL) selection experiment.

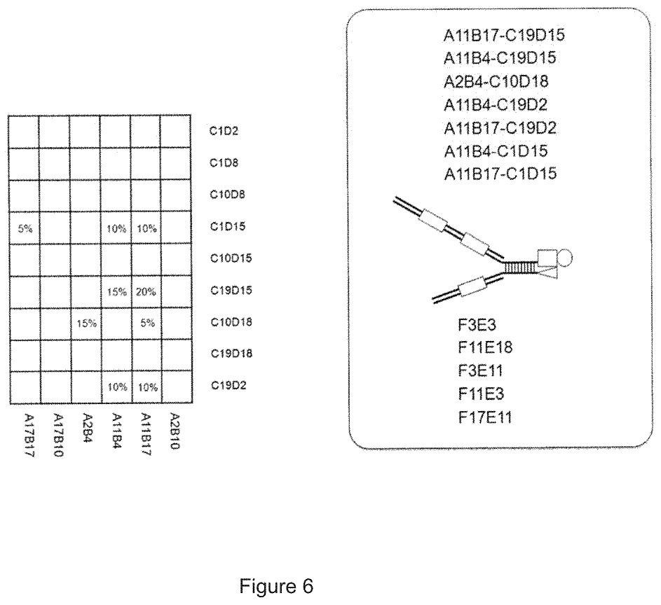

FIG. 6 shows a part of qPCR matrices obtained after a qPCR using a DNA barcode having three coding regions I, II, III as template, 20 different primary primers A and B for coding region I, 20 different primary primers C and D for coding region II, the (same) 20 different primary primers A and D for coding region I to II and 20 different primary primers E and F for coding region III. After having calculated the Value_{coding region I-II}=Value_{matrix-A+D}·Value_{matrix-A+B}·Value_{matrix-C+D}, it has become clear that significant values are obtained for coding regions I-II coded by the nine primer pairings A17B17-C1D15, A2B4-C10D18, A11B4-C1D15, A11B4-C 19D15, A11B4-C19D2, A11B17-C1D15, A11B17-C19D15, A11B17-C10D18 and A11B17-C 19D2. The highest value for coding region III has been determined as well by equation Value_{coding region III}=Value_{matrix-F+E} and it has been found that high values for coding region III are obtained by the five primer pairings F3E3, F11E18, F3E11, F11E3 and F17E11. If the nine coding regions I to II identified above encode a first group of nine different substances and the five coding regions III identified above encode a second group of five different substances, it follows that the combination of the substances of the first group and second group must have been present at a high concentration before the qPCR experiment, i.e. must have been strongly enriched by the (DEL) selection experiment.

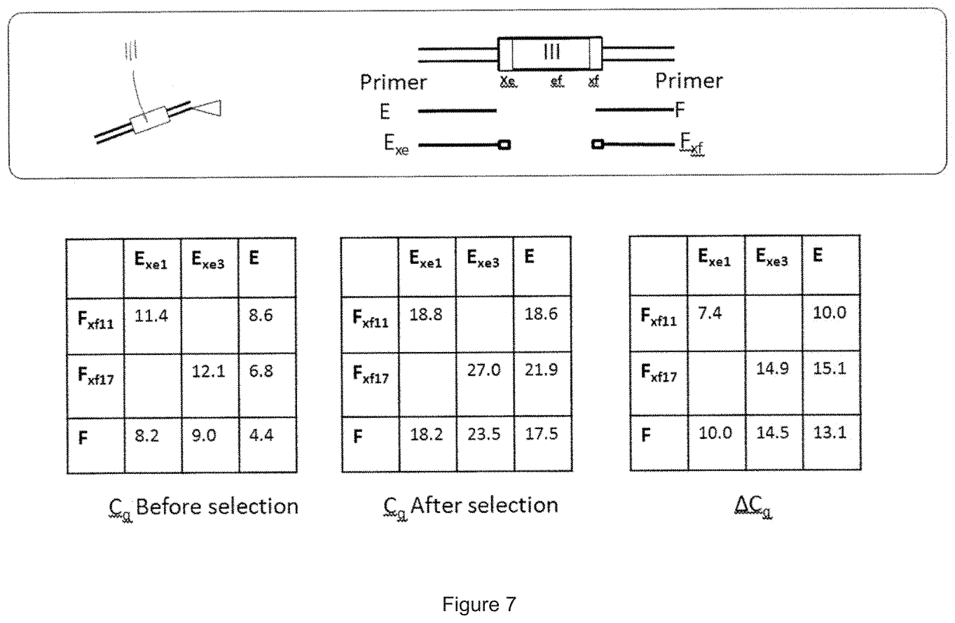

FIG. 7 shows a decoding process for a medium DEL having 306 compounds, each tagged with a DNA barcode. Before and after a DEL selection experiment, a primary qPCR was conducted with the primer pairs E and F, with the primer pairs Eₓₑ and F, with the primer pairs E and F_{xf}, with the primer pairs Eₓₑ₁ and F_{xf11} and with the primer pairs Eₓₑ₃ and F_{xf17}. The obtained C_{q} values before selection are shown in the left matrix, the obtained C_{q} valued after selection are shown in the middle matrix and the ΔC_{q} values are shown in the right matrix in FIG. 7. A ΔCq value of a primer pair which is below the ΔCq value of the primer pair E and F indicates an enrichment of the DNA-substance conjugate. As can be seen in the “ΔC_{q}”-matrix, subcoding region E-F_{xf11} has ΔC_{q}-value of 10.0 which is below the ΔC_{q}-value of 13.1 for subcoding region E-F (i.e. below the control). This means that the subcoding region E-F_{xf11} has been enriched. The same is true for the subcoding region Eₓₑ₁-F with its ΔC_{q}-value of 10.0 being below the ΔC_{q}-value of 13.1 for subcoding region E-F (i.e. being below the control). Thus, the results of the primary qPCR indicate that after the DEL selection experiment, substance(s) encoded by the subcoding regions to which primers Eₓₑ₁ and F_{xf11} bind were enriched more strongly than substances encoded by the subcoding regions to which primers Eₓₑ₃ and F_{xf17} bind. Additionally, for confirmation of said data, a secondary qPCR was conducted with the primer pair Eₓₑ and F_{xf}. Said secondary qPCR confirmed that the subcoding region Eₓₑ₁-F_{xf11} is enriched more strongly than the subcoding region Eₓₑ₃-F_{xf17} (see matrix “ΔC_{q}” in FIG. 7: value in field of column Eₓₑ₁ and row F_{xf11} is much lower than value in field of column Eₓₑ₃ and row F_{xf17} and much lower than the value in the field of column E and row F, which is the control). In summary, both the primary and secondary qPCR demonstrate that the substance(s) connected to the Eₓₑ₁-F_{xf11} subcoding region must have been strongly enriched after the DEL selection experiment.

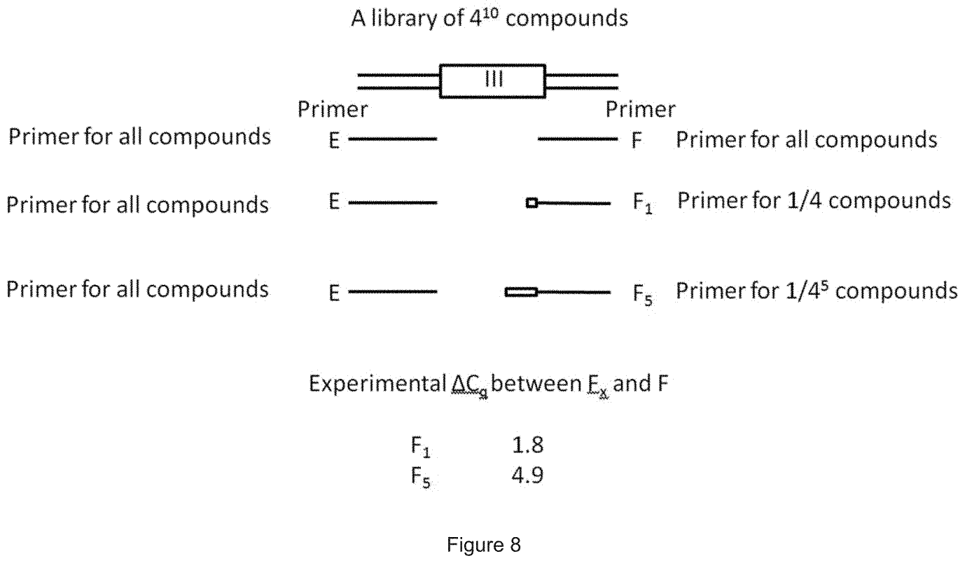

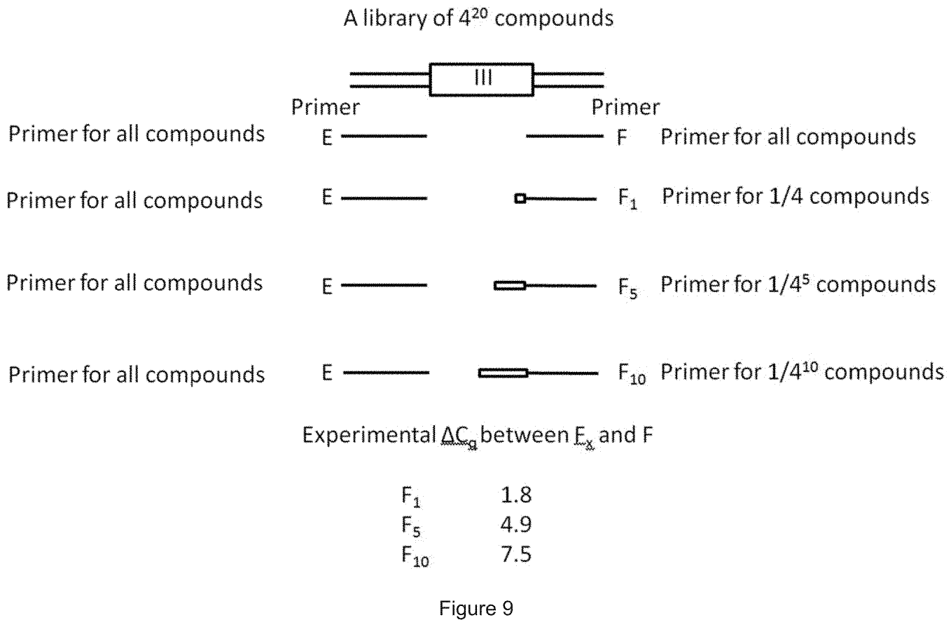

FIG. 8 shows an example of a large DEL having 4¹⁰ compounds, each tagged with a DNA barcode. The libraries were generated by partially degenerated synthesis of DNA. FIG. 8 illustrates the setup for conducting a primary PCR with one (constant) primer E and various different primers Fₙ, wherein each primer Fn codes for a certain subgroup of the library, specifically ¼ⁿ compounds of the library (having 4¹⁰ compounds in total), wherein n is an integer from 0 to 5. This means that if six primers F are used, the first primer Fₒ codes for ¼⁰ of all compounds of the library, i.e. all compounds of the library (=4¹⁰=1048576 compounds), the primer F₁ only codes for ¼ of all compounds of the library (=262144 compounds), the primer F₂ only codes for 1/16 of all compounds of the library (=65536 compounds), the primer F₃ only codes for 1/64 of all compounds of the library (=16384 compounds), the primer F₄ only codes for 1/256 of all compounds of the library (=4096 compounds) and the primer F₅ only codes for 1/1024 of all compounds of the library (=1024 compounds). This means that after the qPCR has been performed, the group of encoded substances which have been selected in the DEL experiment can be significantly narrowed because encoded substances which are not amplifiable with a primer Fₙ, wherein n is 1 to 5, give no signal in qPCR. For example, if the combination of primer E and primer F₁ fails to provide a signal in primary qPCR, it is clear that ¾ of all compounds, i.e. 786432 compounds of 1048576 compounds, are not amplifiable by said qPCR and thus were not enriched by the DEL selection experiment preceding the primary qPCR. Thus, there are only ¼ of all compounds (=262144 compounds) of the DEL library remaining coming into question for having been enriched in the (DEL) selection experiment. FIG. 9 shows another example of a very large DEL having 4²⁰ compounds, each tagged with a DNA barcode. The principal procedure is the same like the one disclosed in FIG. 8 for a DEL having 4¹⁰ compounds. However, due to the larger size of the DEL, it is beneficial if the primary PCR is carried out with more than five different primers F. Specifically, it is beneficial if n is an integer from 0 to 10 in this case. This means that if eleven primers F are used, the first primer Fₒ codes for all compounds of the library and the last primer F₁₀ only codes for ¼¹⁰ of all compounds of the library (=1048576 compounds). This means that after the qPCR has been performed, the group of encoded substances which have been selected in the DEL experiment has been significantly narrowed—For example, a DNA barcode of a DNA-substance conjugate which give no signal with the primer F₁ in qPCR means that said DNA barcode belongs to a group of ¾ of 4²⁰ compounds (≈8.2·10¹¹ compounds) which have not been enriched in the DEL enrichment experiment. Thus, the group of relevant enriched DNA-substance conjugates has been narrowed to ¼ of 4²⁰ compounds (≈2.7·10¹¹ compounds). With each primer Fₙ increasing from n=1 to n=10, the group of relevant compounds is further narrowed. An amplification signal turning up with primer F₁₀ means that the DNA-substance conjugate is within a subgroup of 4¹⁰ (≈1·10⁶ compounds) of 4²⁰ compounds (≈1.1·10¹² compounds) in total.

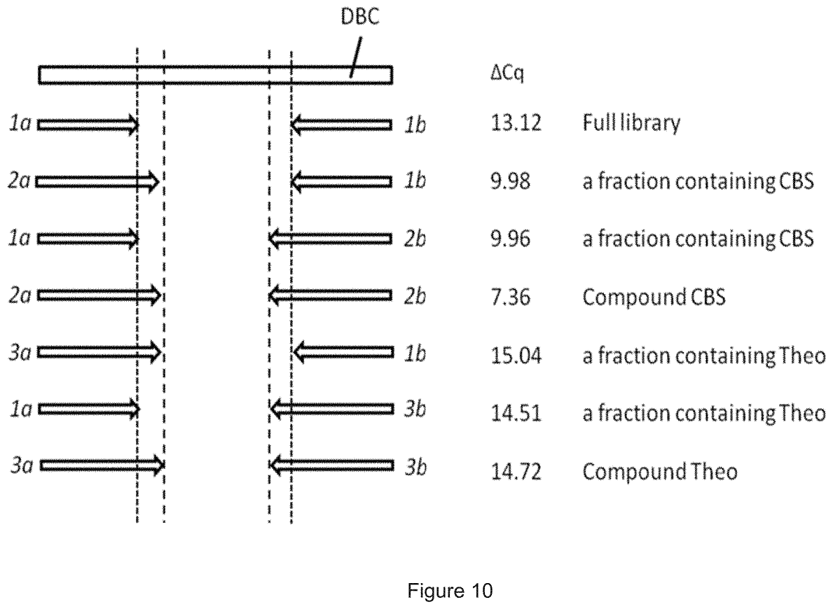

FIG. 10 shows the identification of the substance 4-carboxybenzenesulfonamide (in the following: “CBS”) after a DNA-CBS-conjugate within a DNA-encoded library (DEL) has been enriched by selection with the enzyme carbonic anhydrase II. A small qPCR matrix has been built using three primers 1a, 2a, 3a pairing with the three primers 1b, 2b, 3b. The primer pair 1a, 1b anneals to all DNA-barcodes of the DNA-substance conjugates of the library and thus has the potential to amplify DNA barcodes of the entire library. Primer 2a covers a sub-library containing DNA-CBS-conjugate and primer 2b covers another sub-library containing DNA-CBS-conjugate. The combination of primers 2a and 2b can be assigned exclusively to CBS. Primer 3a covers a sub-library containing theobromine (in the following: “Theo”) conjugated to a DNA barcode (=DNA-Theo-conjugate), primer 3b covers another sub-library containing the DNA-Theo-conjugate. The combination of primers 3a and 3b can be assigned exclusively to Theo. ΔCq is the difference in qPCR cycle before and after selection. A small number reflects large enrichment. ΔCq(1a-1b)>ΔCq(2a-1b)≈ΔCq(1a-2b)>ΔCq(2a-2b) indicated that CBS is remarkably enhanced. ΔCq(1a-1b)<ΔCq(3a-1b)≈ΔCq(1a-3b)=ΔCq(3a-3b) indicated that Theo is not enriched.

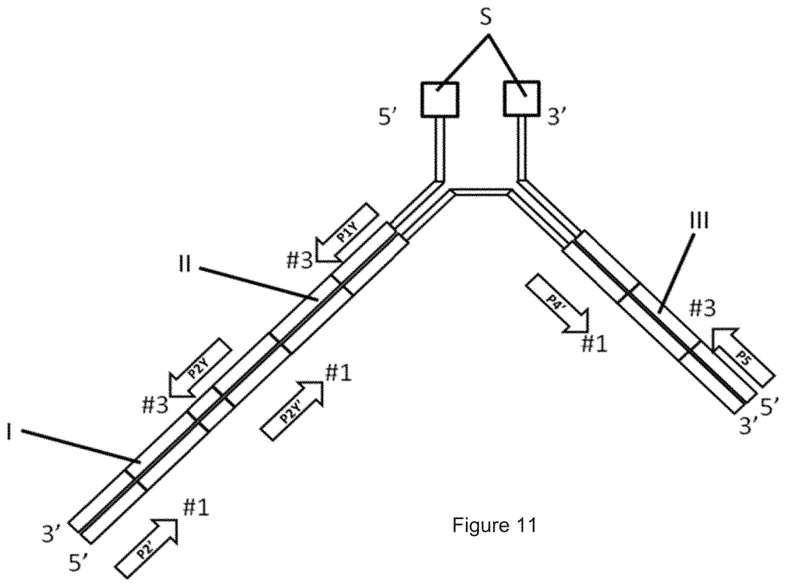

FIG. 11 shows a DNA-substance conjugate in which a first substance S is conjugated chemically covalently to a first coding region DNA sequence I and second coding region DNA sequence and in which a second substance S is conjugated chemically covalently to a third coding region DNA sequence III. Each coding region DNA sequence I, II, III has a first part #1 and a third part #3 to which certain primers can bind (i.e. anneal during qPCR). Primer P2′ (5′-gctgttccca cattgcgt-3′, SEQ-ID Nr. 1) binds to the first part #1 of first coding region DNA sequence I, primer P2Y (5′-ccttctggat tcggtcggag caccatc-3′, SEQ-ID Nr. 2) binds to the third part #3 of first coding region DNA sequence I, primer P2Y′ (5′-gatggtgctc cgaccgaatc cagaagg-3′, SEQ-ID Nr. 3) binds to the first part #1 of second coding region DNA sequence II, primer P1Y (5′-ggaggtgtag acgacagagt atttgactgt cagg-3′, SEQ-ID Nr. 4) binds to the third part #3 of second coding region DNA sequence II, primer P4′ (5′-cagatcgagc aactccac-3′, SEQ-ID Nr. 5) binds to the first part #1 of third coding region DNA sequence III and primer P5 (5′-tggtctcagc cgccctat-3′, SEQ-ID Nr. 6) binds to the third part #3 of third coding region DNA sequence III. If substance S has been enriched after a selection experiment with the DNA-encoded library, amplification with primer pair P2′ and P2Y, primer pair P2Y′ and P1Y and primer pair P4′ and P5 each gives a strong amplification signal in qPCR which allows identification of substance S.

### EXAMPLE 1—DEL COMPRISING DNA BARCODES WITH ONE SINGLE CODING REGION

For DNA codes containing only one single coding region, each code has 3 parts, #1 (first part), #2 (second part) and #3 (third part). Each #2 sequence is a unique code, while each combination of #1 and #3 can also represent a unique code (see e.g. FIG. 1A). Therefore, a sequence of #2 is corresponding to a combination of #1 and #3.

For each part, there is a minimal difference number n between any pair of sequences (e.g. between two different #1 sequences), while n should be ≥2.

### EXAMPLE 2—DEL COMPRISING DNA BARCODES WITH TWO CODING REGIONS

For DNA codes containing two coding regions, each sub-code has 4 parts, for example the first coding region #1 (first part), #2 (second part), #3 (third part) and #4 (fourth part) and the second coding region #1 (first part), #2 (second part), #3 (third part) and #4 (fourth part) (see e.g. FIG. 1B). Each #2 (second part) sequence is a unique sub-code, while each combination of #1, #3 and #4 is corresponding to #2 and each combination of #1, #3 and #4 is corresponding to #2.

For each part, there is a minimal difference number n between any pair of sequences (e.g. between two different #1 sequences), while n should be ≥2.

### EXAMPLE 3—DEL COMPRISING DNA BARCODES WITH MORE THAN TWO CODING REGIONS

For DNA codes containing more than two coding regions (see e.g. FIG. 1B or FIG. 2), two sub-codes at both ends are designed according Example 2, and the sub-code(s) in between is/are designed according to either Example 1, Example 2 or Example 4. It is very unlikely that a high quality DEL can be synthesized using the split-and-pool method. Therefore, a DEL containing less than 4 sub-codes is favorable.

### EXAMPLE 4—DEL COMPRISING DNA BARCODES WITH 5 SUB-CODES

The DNA barcodes of this DEL have 5 parts, #1 (first part), #2 (second part), #3 (third part), #4 (fourth part) and #5 (fifth part).

Each #2 sequence (second part) is a unique sub-code, while each combination of #1 and #3 can also represent a unique sub-code. Therefore, a sequence of #2 is corresponding to a combination of #1 and #3. Each combination of #1 and #4 can also represent a unique sub-code. Therefore, a sequence of #2 is corresponding to a combination of #1 and #4. Each combination of #1 and #5 can also represent a unique sub-code. Therefore, a sequence of #2 is corresponding to a combination of #1 and #5.

For each part, there is a minimal difference number n between any pair of sequences (e.g. between two different #1 sequences), while n should be ≥2 in all designs.

### EXAMPLE 5—DESCRIPTION OF DECODING PROCESS: DECODING ONE-(SUB)-CODE

A primary qPCR matrix is built for the first coding region I using primer A with u different primers B-xb, and primer B with v different primers A-xa. Therefore, the size of resulting matrix is u·v (see e.g. FIG. 3). Same matrices can also be built for the second coding region II and the third coding region III.

A secondary qPCR matrix is built for the first coding region I using pairs of B-xb and A-xa, while B-xb and A-xa are chosen according to the signal intensity in the primary matrix. Same secondary matrices can be built for the second coding region II and the third coding region III. The ranking for each building block can thus be concluded.

For sequence containing two sub-codes, an additional secondary qPCR matrix can be built using A-xa and D-xd, while A-xa and D-xd are chosen according to the signal intensity in the primary matrices.

In combination with the two sub-code matrices (A-xa+B-xb and C-xc+D-xd), the ranking of the combinations can be concluded based on certain algorithm, for example:

Valueⁱ=Valueⁱ_{matrix-A+D}·Valueⁱ_{matrix-A+B}·Valueⁱ_{matrix-C+D},

wherein the Valueⁱ is a value relating and being proportional to the amount of a certain DNA barcode in the DEL. In other words, said Valueⁱ relates to an individual DNA sequence (barcode structure) which resulted from the combinatorial synthesis through joining two building blocks and two sub-codes.

To further validate the Valueⁱ ranking, an additional tertiary qPCR matrix can be built using A-xa-ya and D-xd-yd, while A-xa-ya and D-xd-yd are chosen according to the signal intensity in the primary and secondary matrices and the resulting Valueⁱ ranking.

A full matrix can also be built using A, D and all A-xa-ya and D-xd-yd, though it will be significantly more expensive than the method described before.

The method cannot provide a fully quantitative decoding solution for DEL containing more than two sub-codes. However, combining various primary, secondary, and tertiary rtPCR matrices can provide a Valueⁱ for certain compounds i, which is corresponding to a DNA code containing several sub-codes. All forward and backward primers can be combined to build a matrix.

For example, any primer A, A-xa, A-xa-ya can be combined with any primer B, B-xb, N, N-yn, N-xn-yn, D, D-xd, D-xd-yd to build QPCR matrices. A value for a particular compound can be calculated according to certain algorithm, for example:

Valueⁱ=log₁₀(Valueⁱ_{matrix-A+D}·Valueⁱ_{matrix-A+N}·Valueⁱ_{matrix-M+D}·Valueⁱ_{matrix-A+B}·Valueⁱ_{matrix-C+D}·Valueⁱ_{matrix-M+N})

in which the Valueⁱ_{matrix-A+D}, Valueⁱ_{matrix-A+N} and Valueⁱ_{matrix-M+D} can be either from the secondary, or tertiary matrices, or as a combination of them, and in which the Valueⁱ_{matrix-A+B}·Valueⁱ_{matrix-C+D}·Valueⁱ_{matrix-M+N} are from the secondary matrices.

### LIST OF REFERENCE SIGNS

DBC: DNA barcode sequence;
S: substance;
I: first coding region DNA sequence;
II: second coding region DNA sequence;
II: third coding region DNA sequence;
#1: first part of a coding region DNA sequence;
#2: second part of a coding region DNA sequence;
#3: third part of a coding region DNA sequence;
#4: fourth part of a coding region DNA sequence;
#5: fifth part of a coding region DNA sequence;
A, B, C, D, E, F, M, N: primary primer;
Aₓₐ, B_{xb}, C_{xc}, D_{xd}, Eₓₑ, F_{xf}, Mₓₘ, N_{yn}: secondary primer;
A_{xaya}, D_{xdyd}, M_{xmym}, N_{xnyn}: tertiary primer;
1a, 1b: primary primer binding to all DBS;
2a, 2b: secondary primer binding to DBS of CBS only;
3a, 3b: secondary primer binding to Theo only;
P2′: primer annealing to first part #1 of coding region I;
P2Y: primer annealing to third part #3 of coding region I;
P2Y′: primer annealing to first part #1 of coding region II;
P1Y: primer annealing to third part #3 of coding region II;
P4′: primer annealing to first part #1 of coding region III;
P5: primer annealing to third part #3 of coding region III.

1-15. (canceled)

16. A method for providing a DNA-encoded library, comprising
a) synthesizing many different DNA molecules which differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides; and
b) bonding each of the many different DNA molecules to at least a specific substance forming different DNA-substance conjugates, wherein the DNA-substance conjugates differ from each other by the specific substance and by their DNA molecules;
wherein the first part and the third part encode information regarding the second part of the first coding region, wherein a certain first part and/or a certain third part uniquely codes for a certain group of DNA-substance conjugates which is smaller than the group of all DNA-substance conjugates in the DNA-encoded library.

17. The method according to claim 16, wherein
i) the first coding region DNA sequence comprises at least a fourth part, wherein the second part is located between the fourth and third part and wherein both the combination of the first part and the fourth part and the combination of the first part and the third part of the first coding region encode information about the second part of the first coding region; and
ii) each barcode sequence comprises at least a second coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part of the second coding region encode information about the second part of the second coding region;
wherein a certain combination of a first part and fourth part in a certain coding region uniquely codes for a certain group of DNA-substance conjugates which is smaller than the group of all DNA-substance conjugates which is encoded by the first part alone.

18. The method according to claim 16, wherein
i) each barcode sequence comprises at least a second coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part of the second coding region encode information about the second part of the second coding region; and
ii) each barcode sequence comprises at least a third coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and third part and the of the third coding region encode information about the second part of the third coding region;
wherein a certain combination of a first part and fourth part in a certain coding region uniquely codes for a certain group of DNA-substance conjugates which is smaller than the group of DNA-substance conjugates which is encoded by the first part.

19. The method according to claim 16, wherein
at least one coding region DNA sequence comprises at least a first part, a second part, a third part, a fourth part and a fifth part, wherein the second part is located between the fourth and fifth part and the second part differs between all the DNA molecules by at least two nucleotides,
wherein the combination of the first part and the fourth part and the combination of the fifth part and the third part of the coding region encode information about the second part of the coding region, preferably of all coding regions,
wherein a certain combination of a first part and fourth part uniquely codes for a certain group of DNA-substance conjugates which is smaller than the group of DNA-substance conjugates which is encoded by the first part alone, and
wherein a certain combination of a fifth part and third part uniquely codes for a certain group of DNA-substance conjugates which is smaller than the group of DNA-substance conjugates which is encoded by the third part alone.

20. A DNA-encoded library, comprising many different DNA-ligand conjugates, wherein the DNA-ligand conjugates differ from each other by their ligand and by their DNA molecules,
wherein the DNA molecules of the DNA-ligand conjugates differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides;
wherein the first part and the third part encode information regarding the second part of the first coding region, wherein a certain first part and/or a certain third part uniquely codes for a certain group of DNA-ligand conjugates which is smaller than the group of all DNA-ligand conjugates in the DNA-encoded library.

21. The DNA-encoded library according to claim 20, wherein
i) the first coding region DNA sequence comprises at least a fourth part, wherein the second part is located between the fourth and third part and wherein both the combination of the first part and the fourth part and the combination of the first part and the third part of the first coding region encode information about the second part of the first coding region; and
ii) each barcode sequence comprises at least a second coding region DNA sequence comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part of the second coding region encode information about the second part of the second coding region;
wherein a certain combination of a first part and fourth part in a certain coding region uniquely codes for a certain group of DNA-ligand conjugates which is smaller than the group of all DNA-ligand conjugates which is encoded by the first part alone.

22. The DNA-encoded library according to claim 21, wherein each barcode sequence comprises at least a third coding region DNA sequence, which is on the same DNA strand as the second coding region, comprising at least a first part, a second part, a third part, and a fourth part, wherein the second part is located between the fourth and third part and the second part differs between all the DNA molecules by at least two nucleotides, wherein both the combination of the first part and the fourth part and the combination of the first part and the third part and the of the third coding region encode information about the second part of the third coding region, wherein a certain combination of a first part and fourth part in the second coding region and in the third coding region uniquely codes for a certain group of DNA-ligand conjugates which is smaller than the group of DNA-ligand conjugates which is encoded by the first part alone.

23. The DNA-encoded library according to claim 20, wherein at least one coding region DNA sequence comprises at least a first part, a second part, a third part, a fourth part and a fifth part, wherein the second part is located between the fourth and fifth part and the second part differs between all the DNA molecules by at least two nucleotides,
wherein the combination of the first part and the fourth part and the combination of the fifth part and the third part of the coding region encode information about the second part of the coding region, and
wherein a certain combination of a first part and fourth part uniquely codes for a certain group of DNA-ligand conjugates which is smaller than the group of DNA-ligand conjugates which is encoded by the first part alone and
wherein a certain combination of a fifth part and third part uniquely codes for a certain group of DNA-ligand conjugates which is smaller than the group of DNA-ligand conjugates which is encoded by the third part alone.

24. A method of decoding a DNA-encoded library according to claim 20, comprising
a) performing a qPCR with the DNA-encoded library, wherein the following primers are utilized:
a primer A and a primer B for amplifying the first coding region of every DNA-ligand conjugate; and
many different primers A-xN which anneal to the different first parts of the first coding region and many different primers B-yN which anneal to the different third parts of the first coding region, wherein primer A-xN has an identical length like the coding region primer A by shortening x nucleotides at its 5′-end, primer B-yN has an identical length like the coding region primer B by shortening y nucleotides at its 5′-end, N represents a A, T, G or C and x and y represent the total number of any one of A, T, G or C at the 3′-end of the primers, wherein x is an integer from 2 to 6;
b) calculating a mathematical product of the signal value of each primer A-xN and each primer B-xN by following equation:
Value (A-xN)ᵢ=signal value [(A-xN)ᵢ+B]·signal value [(A-xN)ᵢ+(B-xN)ᵢ];
and
Value (B-yN)ᵢ=signal value [(B-yN)ᵢ+A]·signal value [(B-yN)ᵢ+(A-xn)ᵢ],
wherein i is an integer and defines a specific primer, and the “+”-sign indicates a combination of two primers; wherein signal value is the percentage of abundance related to the whole set of qPCR quantification using different primers annealed to the same region; and
c) comparing the obtained mathematical products for each of the primers (A-xN)ᵢ and (B-yN)ᵢ, wherein those primers with high values code for DNA-ligand conjugates which are present at a high concentration in the DNA-encoded library.

25. The method according to claim 24, wherein the method comprises
i) calculating a mathematical product of the value obtained for each primer A-xN and each primer B-yN by following equation Value (A−B)ᵢ=Value (A-xN)ᵢ·Value (B-yN)ᵢ;
ii) comparing the obtained mathematical products for each of the combination of primers (A-xN)ᵢ and (B-yN)ᵢ, wherein those primer combinations with high values code for DNA-ligand conjugates which are present at a high concentration in the DNA-encoded library.

26. The method according to claim 24, wherein the qPCR is performed with a DNA-encoded library as a template,
wherein the DNA-encoded library comprises many different DNA-ligand conjugates, wherein the DNA-ligand conjugates differ from each other by their ligand and by their DNA molecules,
wherein the DNA molecules of the DNA-ligand conjugates differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides;
wherein the first part and the third part encode information regarding the second part of the first coding region, wherein a certain first part and/or a certain third part uniquely codes for a certain group of DNA-ligand conjugates which is smaller than the group of all DNA-ligand conjugates in the DNA-encoded library;
the method comprising:
i) performing a qPCR with the following primers:
a first coding region primer A and a first coding region primer primer B for amplifying the first coding region of every DNA-ligand conjugate; and
many different primers A-xN which anneal to the different first parts, or first and fourth parts of the first coding region and many different primers B-yN which anneal to the different third parts of the first coding region, wherein A-xN has an identical length like the coding region primer A by shortening x nucleotides at its 5′-end, B-yN has an identical length like the coding region primer B by shortening y nucleotides at its 5′-end, N represents a A, T, G or C and x and y represent the total number of any one of A, T, G or C at the 3′-end of the primers, wherein x is an integer from 6 to 10, and y is an integer from 2 to 6; and
a second coding region primer C and a second coding region primer D for amplifying the second coding region of every DNA-ligand conjugate; and
many different primers D-yN which anneal to the different first parts, or first and fourth parts of the second coding region and many different primers C-xN which anneal to the different third parts of the second coding region, wherein primer C-xN has an identical length like the coding region primer C by shortening x nucleotides at its 5′-end, primer D-yN has an identical length like the coding region primer D by shortening y nucleotides at its 5′-end, N represents a A, T, G or C and x and y represent the total number of any one of A, T, G or C at the 3′-end of the primers, wherein x is an integer from 6 to 10, and y is an integer from 2 to 6;
ii) calculating a mathematical product of the signal value of each primer A-xN, each primer B-yN, each primer C-xN and each primer D-yN by following equation:
Value (A-xN)ᵢ=signal value [(A-xN)ᵢ+B]·signal value [(A-xN)ᵢ+(B-xN)ᵢ];
Value (B-yN)ᵢ=signal value [(B-yN)ᵢ+A]·signal value [(B-yN)ᵢ+(A-xN)ᵢ],
Value (C-xN)ᵢ=signal value [(C-xN)ᵢ+D]·signal value [(C-xN)ᵢ+(D-xn)ᵢ],
Value (D-yN)ᵢ=signal value [(D-yN)ᵢ+C]·signal value [(D-yN)ᵢ+(C-xn)ᵢ],
wherein i is an integer and defines a specific primer, and the “+”-sign indicates a combination of two primers; wherein signal value is the percentage of abundance related to the whole set of qPCR quantification using different primers annealed to the same region; and
iii) comparing the obtained mathematical products for each of the primers (A-xN)ᵢ, (B-yN)ᵢ, (C-xN)ᵢ and (D-yN)ᵢ, wherein those primers with high values code for DNA-ligand conjugates which are present at a high concentration in the DNA-encoded library.

27. The method according to claim 26, wherein the method comprises
i) calculating a mathematical product of the value obtained for each primer A-xN and each primer B-yN, for each primer A-xN and each primer D-xN and for each primer C-yN and D-xN by following equation
Value (A−B)ᵢ=Value (A-xN)ᵢ·Value (B-yN)ᵢ;
Value (A−D)ᵢ=Value (A-xN)ᵢ·Value (D-yN)ᵢ;
Value (C−D)ᵢ=Value (C-xN)ᵢ·Value (D-yN)ᵢ;
ii) calculating the mathematical product of the Value (A−B)ᵢ, (A−D)ᵢ and (C−D)ᵢ for each primer i by the following equation Valueⁱ=value (A−B)ᵢ·value (A−D)ᵢ·value (C−D)ᵢ
iii) comparing the obtained mathematical products Valueⁱ, wherein those primer combinations i with high values code for DNA-ligand conjugates which are present at a high concentration in the DNA-encoded library.

28. The method according to claim 24, wherein the qPCR is performed with a DNA-encoded library,
wherein the DNA-encoded library comprises many different DNA-ligand conjugates, wherein the DNA-ligand conjugates differ from each other by their ligand and by their DNA molecules,
wherein the DNA molecules of the DNA-ligand conjugates differ from each other by comprising different DNA barcode sequences, wherein each DNA barcode sequence comprises at least a first coding region DNA sequence comprising at least a first part, a second part and a third part, wherein the second part is located between the first and third part and the second part differs between all the DNA molecules by at least two nucleotides;
wherein the first part and the third part encode information regarding the second part of the first coding region, wherein a certain first part and/or a certain third part uniquely codes for a certain group of DNA-ligand conjugates which is smaller than the group of all DNA-ligand conjugates in the DNA-encoded library;
the method comprising:
i) performing a qPCR with the following primers:
a first coding region primer A and a first coding region primer B for amplifying the first coding region of every DNA-ligand conjugate; and
many different primers A-xN which anneal to the different first parts, or first and fourth parts of the first coding region, and many different primers B-yN which anneal to the different third parts of the first coding region, wherein A-xN has an identical length like the coding region primer A by shortening x nucleotides at its 5′-end, B has an identical length like the coding region primer B-yN by shortening y nucleotides at its 5′-end, N represents a A, T, G or C and x and y represent the total number of any one of A, T, G or C at the 3′-end of the primers, wherein x is an integer from 6 to 10, preferably 8, and y is an integer from 2 to 6, preferably 4; and
a second coding region primer C and a second coding region primer D for amplifying the second coding region of every DNA-ligand conjugate; and
many different primers D-yN which anneal to the different first parts, or first and fourth parts of the second coding region and many different primers C-xN which anneal to the different third parts of the second coding region, wherein primer C-xN has an identical length like the coding region primer C by shortening x nucleotides at its 5′-end, primer D-yN has an identical length like the coding region primer D by shortening y nucleotides at its 5′-end, N represents a A, T, G or C and x and y represent the total number of any one of A, T, G or C at the 3′-end of the primers, wherein x is an integer from 6 to 10, and y is an integer from 2 to 6;
a third coding region primer E and a third coding region primer F for amplifying the third coding region of every DNA-ligand conjugate; and
many different primers E-xN which anneal to the different first parts of the third coding region and many different primers F-yN which anneal to the different third parts of the third coding region, wherein primer E-xN has an identical length like the coding region primer E by shortening x nucleotides at its 5′-end, primer F-yN has an identical length like the coding region primer F by shortening y nucleotides at its 5′-end, N represents a A, T, G or C and x and y represent the total number of any one of A, T, G or C at the 3′-end of the primers, wherein x is an integer from 6 to 10, and y is an integer from 2 to 6;
ii) calculating a mathematical product of the signal value of each primer A-xN, each primer B-yN, each primer C-xN, each primer D-yN, each primer E-xN and each primer F-yN by following equation:
Value (A-xN)ᵢ=signal value [(A-xN)ᵢ+B]·signal value [(A-xN)ᵢ+(B-xN)ᵢ];
Value (B-yN)ᵢ=signal value [(B-yN)ᵢ+A]·signal value [(B-yN)ᵢ+(A-xN)ᵢ],
Value (C-xN)ᵢ=signal value [(C-xN)ᵢ+D]·signal value [(C-xN)ᵢ+(D-xN)ᵢ],
Value (D-yN)ᵢ=signal value [(D-yN)ᵢ+C]·signal value [(D-yN)ᵢ+(C-xN)ᵢ],
Value (E-xN)ᵢ=signal value [(E-xN)ᵢ+F]·signal value [(E-xN)ᵢ+(F-xn)ᵢ],
Value (F-yN)ᵢ=signal value [(F-yN)ᵢ+E]·signal value [(F-yN)ᵢ+(E-xN)ᵢ],
wherein i is an integer and defines a specific primer, and the “+”-sign indicates a combination of two primers; wherein signal value is the percentage of abundance related to the whole set of qPCR quantification using different primers annealed to the same region; and
iii) comparing the obtained mathematical products for each of the primers (A-xN)ᵢ, (B-yN)ᵢ, (C-xN)ᵢ, (D-yN)ᵢ, (E-xN)ᵢ and (N-yN)ᵢ, wherein those primers with high values code for DNA-ligand conjugates which are present at a high concentration in the DNA-encoded library.

29. The method according to claim 28, wherein the method comprises
i) calculating a mathematical product of the value obtained for each primer A-xN and each primer B-yN, for each primer A-xN and each primer D-xN, for each primer C-yN and D-xN, for each primer A-xN and N-yN, for each primer M-xN and D-yN and for each primer M-xN and N-yN by following equation
Value (A−B)ᵢ=Value (A-xN)ᵢ·Value (B-yN)ᵢ;
Value (A−D)ᵢ=Value (A-xN)ᵢ·Value (D-yN)ᵢ;
Value (C−D)ᵢ=Value (C-xN)ᵢ·Value (D-yN)ᵢ;
Value (A−F)ᵢ=Value (A-xN)ᵢ·Value (F-yN)ᵢ;
Value (E−D)ᵢ=Value (E-xN)ᵢ·Value (D-yN)ᵢ;
Value (E−F)ᵢ=Value (E-xN)ᵢ·Value (F-yN)ᵢ;
ii) calculating the mathematical product of the values (A−B)ᵢ, (A−D)ᵢ, (C−D)ᵢ, (A-F)ᵢ, (E-D)ᵢ and (E-F)ᵢ for each primer combinations i by the following equation
Valueⁱ=value (A−B)ᵢ·value (A−D)ᵢ·value (C−D)ᵢ·value (A−F)ᵢ·value (E−D)ᵢ·value (E−F)ᵢ;
iii) comparing the obtained mathematical products Valueⁱ, wherein those primer combinations i with high values code for DNA-ligand conjugates which are present at a high concentration in the DNA-encoded library.

30. The method according to claim 29, wherein the method further comprises calculating a Valueⁱ by the following calculation:
Valueⁱ=log₁₀[value (A−B)ᵢ value (A−D)ᵢ value (C−D)ᵢ value (A−F)ᵢ·value (E−D)ᵢ·value (E−F)ᵢ].


SEQUENCE LISTING (6 sequences)

SEQ ID NO: 1 - DNA, 18 bp, Artificial Sequence
  primer annealing to first part 1 of coding region I
gctgttcccacattgcgt

SEQ ID NO: 2 - DNA, 27 bp, Artificial Sequence
  primer annealing to third part 3 of coding region I
ccttctggattcggtcggagcaccatc

SEQ ID NO: 3 - DNA, 27 bp, Artificial Sequence
  primer annealing to first part 1 of coding region II
gatggtgctccgaccgaatccagaagg

SEQ ID NO: 4 - DNA, 34 bp, Artificial Sequence
  primer annealing to third part 3 of coding region II
ggaggtgtagacgacagagtatttgactgtcagg

SEQ ID NO: 5 - DNA, 18 bp, Artificial Sequence
  primer annealing to first part 1 of coding region III
cagatcgagcaactccac

SEQ ID NO: 6 - DNA, 18 bp, Artificial Sequence
  primer annealing to third part 3 of coding region III
tggtctcagccgccctat